(19) United States
(12) Patent Application Publication
(10) Pub. No.: US 2026/0286327 A1
(43) Pub. Date: Sep. 24, 2026

(54) SYSTEMS AND METHODS USING NUCLEASES MODIFIED WITH HALOGENATED AMINO ACIDS

(71) Applicant: Colorado State University Research Foundation, Fort Collins, CO (US)

(72) Inventors: Pui Shing Ho, Fort Collins, CO (US); Cesar G. Mendez, Fort Collins, CO (US); Margaret G. Walker, Fort Collins, CO (US); Alexander Ho, Fort Collins, CO (US); Ryan S. Czarny, Fort Collins, CO (US); Anthony K. Rappe, Fort Collins, CO (US)

(21) Appl. No.: 19/570,784

(22) Filed: Mar. 18, 2026

Related U.S. Application Data
(60) Provisional application No. 63/773,741, filed on Mar. 18, 2025.

(51) Int. Cl. C12N 9/22

(52) U.S. Cl. CPC C12N 9/22; C12Y 301/30

(57) ABSTRACT

The present disclosure provides methods, compositions, and systems related to modulating the catalytic activity of nucleases (e.g., endonucleases) through site-specific incorporation of halogenated unnatural amino acids, and cleaving target nucleic acids using such modified nucleases under controlled pH conditions.

### CROSS-REFERENCE TO RELATED APPLICATIONS

This application claims the benefit of U.S. Provisional Application No. 63/773,741, filed Mar. 18, 2025, the content of which is herein incorporated by reference in its entirety.

### STATEMENT REGARDING FEDERALLY SPONSORED RESEARCH DEVELOPMENT

This invention was made with government support under grants 2124202 and 2203161 awarded by the National Science Foundation. The government has certain rights in the invention.

### FIELD

The present disclosure provides methods, compositions, and systems related to modulating the catalytic activity of nucleases (e.g., endonucleases) through site-specific incorporation of halogenated unnatural amino acids, and cleaving target nucleic acids using such modified nucleases under controlled pH conditions.

### SEQUENCE LISTING STATEMENT

The content of the electronic sequence listing titled CSU 44849_202_SequenceListing.xml (Size: 5,151 bytes; and Date of Creation: Mar. 17, 2026) is herein incorporated by reference in its entirety.

### BACKGROUND

Nature employs a limited set of chemical elements and catalytic strategies to generate the full diversity of functional biomolecules observed in living systems. In recent years, synthetic biology and enzyme engineering have begun to transcend these natural boundaries, creating proteins and other biomolecules with functions and properties not found in nature. Central to this effort has been the site-specific incorporation of non-canonical amino acids (ncAAs) into proteins, which introduces chemical functionalities, such as bioorthogonal reactive groups, spectroscopic probes, post-translational modification mimics, and novel noncovalent interaction capabilities, which are unavailable within the standard amino acid repertoire. Complementary advances in directed evolution, computational enzyme design, and expanded genetic code technologies have further accelerated the creation of engineered enzymes with tailored catalytic activities, altered substrate specificities, and switchable or externally controllable functions.

Despite this progress, significant challenges remain in engineering enzymes whose catalytic activity can be precisely and reversibly modulated by simple environmental stimuli. Many naturally occurring enzymes, including nucleases that are widely used in molecular biology and biotechnology, depend on divalent metal ion cofactors for activity, which constrain the conditions under which they can operate and limit the degree of temporal control that can be exerted over their function. There is accordingly a need for engineered enzymes that exploit novel noncovalent interactions to achieve catalytic mechanisms that are responsive to readily adjustable parameters such as pH, thereby enabling new modes of activity control that are not accessible with conventional enzyme engineering approaches.

### SUMMARY

Embodiments of the present disclosure include methods for modulating the catalytic activity of a modified endonuclease.

In some embodiments, the methods comprise: contacting the modified endonuclease with a target nucleic acid in a reaction medium, wherein the modified endonuclease comprises a halogenated amino acid substituted at a position corresponding to a divalent metal ion-coordinating residue of a parent endonuclease and the halogenated amino acid forms a halogen bond with the target nucleic acid; and adjusting the reaction medium to a pH where the modified endonuclease cleaves the target nucleic acid.

In some embodiments, the halogenated amino acid is a halogenated tyrosine. In some embodiments, the halogenated tyrosine is a meta-halotyrosine. In some embodiments, the halogenated tyrosine is meta-iodotyrosine.

In some embodiments, the parent endonuclease is endonuclease G (EndoG) or a homolog or ortholog thereof.

In some embodiments, the pH where the modified endonuclease cleaves the target nucleic acid is below 7.0 or above 8.0.

In some embodiments, the method further comprises adjusting the pH of the reaction medium to a pH which reduces the catalytic activity of the modified endonuclease.

In some embodiments, the method comprises sequentially adjusting the pH of the reaction medium one or more times between a pH where the modified endonuclease cleaves the target nucleic acid and a pH which reduces the catalytic activity of the modified endonuclease.

In some embodiments, the pH which reduces the catalytic activity of the modified endonuclease is 7.0 to 8.0.

In some embodiments, the target nucleic acid is DNA.

In some embodiments, the pH of the reaction medium is below 7.0. In some embodiments, the reaction medium is substantially free of divalent metal cofactor. In some embodiments, the modified endonuclease retains catalytic activity in the presence of a metal chelator at a concentration sufficient to substantially inactivate the parent endonuclease.

In some embodiments, the pH of the reaction medium is above 8.0. In some embodiments, the reaction medium comprises a divalent metal cofactor. In some embodiments, the divalent metal cation is Mg²⁺.

Other embodiments of the present disclosure include methods for cleaving a target nucleic acid. In some embodiments, the methods comprise: providing a modified endonuclease comprising a halogenated amino acid substituted at a position corresponding to a divalent metal ion-coordinating residue of a parent endonuclease, wherein the halogenated amino acid forms a halogen bond with a target nucleic acid; and contacting the target nucleic acid with the modified endonuclease at a select pH where the modified endonuclease cleaves the target nucleic acid.

In some embodiments, the select pH is below 7.0 or above 8.0. In some embodiments, the select pH is from 5.5 to 7.0 or from 8.0 to 10.0.

In some embodiments, the modified endonuclease exhibits reduced catalytic activity at a pH of 7.0 to 8.0 relative to activity at a pH below 7.0 or above 8.0.

In some embodiments, the method comprises adjusting the pH one or more times between a pH wherein the modified endonuclease exhibits reduced catalytic activity and the select pH.

In some embodiments, the select pH is below 7.0. In some embodiments, the method does not comprise contacting the modified endonuclease and/or target nucleic acid with a divalent metal cofactor. In some embodiments, the modified endonuclease retains catalytic activity in the presence of a metal chelator at a concentration sufficient to substantially inactivate the parent endonuclease.

In some embodiments, the select pH is above 8.0. In some embodiments, the method further comprises contacting the modified endonuclease and/or target nucleic acid with a divalent metal cofactor. In some embodiments, the divalent metal cation is Mg²⁺.

In some embodiments, the halogenated amino acid is a halogenated tyrosine. In some embodiments, the halogenated tyrosine is a meta-halotyrosine. In some embodiments, the halogenated tyrosine is meta-iodotyrosine.

In some embodiments, the target nucleic acid is DNA. In some embodiments, the target nucleic acid is in a reaction medium.

In some embodiments, the parent endonuclease is endonuclease G (EndoG) or a homolog or ortholog thereof.

Other embodiments and embodiments of the disclosure will be apparent in light of the following detailed description and related figures.

### BRIEF DESCRIPTION OF THE DRAWINGS

FIGS. 1A-1F: Structure and Catalytic Activities of mEndoG Constructs. FIG. 1A is a crystal structure of the mEndoG homodimer, with the T₅ DNA from the C. elegans CPS6 ortholog of EndoG docked into the active site of the right-side monomer. FIG. 1B is a schematic for the mechanism of mEndoG, showing glutamate E136 coordinated to the catalytic Mg²⁺ and histidine H97 serving as a general base to deprotonate the nucleophilic water. FIG. 1C shows the active site in WT-mEndoG shown with Mg²⁺ coordinated (dashed lines) to the carboxylate oxygens of E136 through bridging waters and the carbonyl oxygen of asparagine N128. FIG. 1D shows a model of the E136 replaced by ^{mI}Y136, generating the ^{mI}Y-mEndoG construct. This RotA model positions the iodine to potentially form X-bonds to the scissile phosphate oxygens of the DNA. Asparagine N103 is shown as an H-bond donor to bridge ^{mI}Y136 to the protonated histidine H104. FIG. 1E shows the RotB model of ^{mI}Y-mEndoG construct which positions the iodine away from the DNA phosphate oxygens and N103. FIG. 1F shows the junction substrate and DNA cutting activities of non-halogenated and halogenated mEndoG constructs. Schematic of the DNA junction substrate (top left; formed with polynucleotides of SEQ ID NOs: 1-4), showing the 5-hydroxymethylcytosine base (^{5hm}C, bold, outlined text) position and the strands labeled at their 5′-ends with cyanine-5 (Cy5, bottom star) or fluorescein (FITC, top star) fluorophores. Duplex DNA substrates result from incomplete formation of junctions from the sequences. Polyacrylamide gel electrophoresis (PAGE, bottom right) analysis of the DNA substrates (four-stranded junction and duplex, lane 1) and products after treatment with WT-mEndoG (lane 2), iodinated ^{mI}Y-mEndoG (lane 3), chlorinated ^{mCI}Y-mEndoG (lane 4), or the nonhalogenated tyrosyl Y-mEndoG (lane 5).

FIGS. 2A-2G: Effects of pH and EDTA on the DNA Cutting Activity of WT- and Halogenated ^{mX}Y-mEndoG. FIG. 2A is a graph of the apparent pseudo-first order kinetic rate constants (k_{APP}) for WT-mEndoG. The pH titration curve (dotted line) was modeled using standard Henderson-Hasselbalch equations (36, 37). The titration curve was modeled with two pKa values (pKa₁=6.78, pKa₂=7.69), with R²=0.91. FIG. 2B shows the quantitation of cleaved DNA products after 30 minutes reaction from halogenated (^{mCI}Y-mEndoG, green bars, and ^{mI}Y-mEndoG, magenta bars) and unhalogenated (grey bars) tyrosine mutant constructs relative to those from WT-mEndoG at pHs from 7.0 to 9.0. FIG. 2C shows the effect of pH on k_{APP} for the base (High pH) facilitated ^{mI}Y-mEndoG activity, analyzed similarly to WT-mEndoG in a. The Henderson-Hasselbalch curve was fit with a pka=8.7 (R²=0.9). FIG. 2D shows the effect of pH on k_{APP} for the acid (Low pH) facilitated ^{mI}Y-mEndoG activity. The Henderson-Hasselbalch curve was fit with a pka=6.98 (R²=0.98). FIG. 2E shows PAGE analysis of junction (J) and duplex (D) substrates and products (P) after treatment with WT-mEndoG or ^{mI}Y-mEndoG in 0 mM, 2 mM, or 4 mM concentrations of EDTA at pH 7.0 for 2- or 4-hours. FIG. 2F shows normalized intensity (<Intensity>) profiles of the scanned gel lanes from e using the NIH ImageJ program (38) for WT-mEndoG and ^{mI}Y-mEndoG after 4 hours catalyzed reactions in 0 mM, 2 mM, and 4 mM EDTA. FIG. 2G is a graph of the percent activity for WT-mEndoG (blue) and ^{mI}Y-mEndoG (magenta) from FIGS. 2F and 8 relative to their respective maximum activities at 0 mM EDTA. Error bars are the standard errors of the mean from at least 3 independent measurements.

FIGS. 3A-3C: H-bond Network to Enhance the ^{mI}Y X-bond and Quantum Mechanical (QM) Analysis of Electrostatic Potentials (ESP) of Models for WT- and ^{mX}Y-mEndoG Active Site Residues. FIG. 3A is a model showing the proposed H104 . . . N103 . . . ^{mI}Y136 network. In this model, protonation of H104 under acidic conditions creates the H-bonded H104-H⁺ . . . O═C—N103-NH₂ . . . . I network that significantly enhances the X-bonding potential of the ^{mI}Y136 iodine substituent. FIG. 3B is a PAGE assay of DNA substrates and products generated by WT-mEndoG, ^{mI}Y-mEndoG and their respective N103A and H104A mutants. FIG. 3C shows a comparison of ESP values for the Mg²⁺ cofactor in WT-mEndoG to those of the halogens in models for the ^{mI}Y-mEndoG active site. The model of Mg²⁺ coordinated to N128 and E136 in WT-mEndoG was constructed as complexes with acetamide and acetate molecules, respectively. All atoms, along with waters were placed according to their respective positions as seen in the crystal structure of WT-mEndoG. The complex included either all four waters from the crystal structure, or with waters removed (leaving 2 or 3 coordinated solvent molecules) that would allow coordination of the Mg²⁺ to the phosphate oxygens of the DNA substrate. ESP were calculated for models of ^{mCI}Y and ^{mI}Y residues alone, in complex with the carbonyl oxygen (N═O) or the amino group (NH₂) of acetamide (model for the N103 side chain) pointed towards the halogen, with neutral imidazole (model for the H104 side chain, labeled H) or protonated imidazole (H⁺) in the network. Values were plotted for the most positive point on the respective ESP surfaces.

FIG. 4: DNA Cutting Activity of the Nonhalogenated E136Y-mEndoG Construct Compared to the Enzyme with E136 Replaced by a para-iodophenylalanine (^{I}F-mEndoG). Left-hand panel shows junction construct for the assay, showing the DNA strands labeled with the Cy5 (red star) or fluorescence (FITC, blue star). The DNA cleavage products were analyzed by a denaturing polyacrylamide gel after 90 minutes of reaction at pH6.5.

FIG. 5: Fluorescence Polarization Titration to Determine Dissociation Constants (K_{D}) of the meta-iodotyrosyl Mouse Endonuclease G (^{mI}Y-mEndoG) Construct.

FIG. 6: Effect of the Metal Chelator Ethylenediaminetetraacetic acid (EDTA at 0 mM, 1 mM, and 2 mM concentrations) on DNA Cutting Activity after 2 hours Incubation with WT-mEndoG or ^{mI}Y-mEndoG under Basic (pH 8.5) Conditions. Normalized intensity (<Intensity>) profiles of the PAGE analyses for WT-mEndoG (blue lines) and ^{mI}Y-mEndoG (magenta lines) are shown in the two right-hand panels. Black dashed lines are the profiles from the DNA only (far left, no enzyme) lane of the gel.

FIG. 7: pH Dependence of DNA Cleavage Rates for ^{mI}Y-mEndoG. Data from one example titration experiment to determine k_{APP} for ^{mI}Y-mEndoG. Scans of intensities from gels with no enzyme (representing the 0 time point) and after 5, 10, 20 and 30 minutes of reaction (left panels) are normalized and plotted as fractions as substrate (red circles) and products (blue squares) for each time period (right panels). The fractions for each time period were treated as pseudo-first-order reactions to determine the k_{APP} values at each pH.

FIG. 8: Denaturing PAGE Gel Analysis on Effects of EDTA (0 mM, 1 mM, and 2 mM concentrations) on DNA Cutting Activity of WT-mEndoG and ^{mX}Y-mEndoG (X=I or Cl) under Neutral (pH=7.0) Conditions. The slow migrating bands in the 0 mM WT-mEndoG and ^{mX}Y-mEndoG Cy5 lanes of the PAGE gel are small fragments that migrate anomalously due to the positive charge of the Cy5 label.

FIG. 9: Quantum Mechanical (QM) Analysis of Electrostatic Potentials (ESP) of Models for WT- and ^{mX}Y-mEndoG Active Site Residues. In this analysis, the top lighter colored bar associated with the ESP calculation for each ^{mX}Y-mEndoG model shows the additional energy associated with the tyrosyl OH rotated towards and H-bonded to the halogen. Comparison of ESP values for the Mg²⁺ cofactor in WT-mEndoG to those of the halogens in models for the ^{mI}Y-mEndoG active site (see main text for description for how each model was constructed). The complex included either all four waters from the crystal structure, or with waters removed (leaving 2 or 3 coordinated solvent molecules) that would allow coordination of the Mg²⁺ to the phosphate oxygens of the DNA substrate. ESP were calculated for models of ^{mCI}Y and ^{mI}Y residues alone, in complex with the carbonyl oxygen (N═O) or the amino group (NH₂) of acetamide (model for the N103 side chain) pointed towards the halogen, with neutral imidazole (model for the H104 side chain, labeled H) or protonated imidazole (H⁺) in the network. Values were plotted for the most positive point on the respective ESP surfaces.

### DETAILED DESCRIPTION

Disclosed herein are methods which utilize a modified nuclease (e.g., an endonuclease) which includes a halogenated amino acid substitution at a position corresponding to a divalent metal ion-coordinating residue. The halogenated amino acid forms a halogen bond with a target nucleic acid, thereby providing an alternative catalytic mechanism that can be controlled by adjusting the pH of the reaction medium. As such, the modified nucleases can be used in methods for cleaving target nucleic acids at select pH values or methods for modulating nuclease activity by adjusting the pH.

A modified enzyme active site was generated in mouse endonuclease G (mEndoG) in which the catalytic magnesium dication (Mg²⁺) is functionally replaced by a halotyrosine ncAA. Halogen bonds (X-bonds) are non-classical noncovalent interactions that are used in chemistry to design novel materials. The X-bond is most readily described by the σ-hole model, where the σ-molecular orbital of the covalent bonded halogen subsumes the electron from the atomic p_{z}-orbital, resulting in an electropositive “σ-hole” at the tip and leaving an electronegative waist around the halogen substituent. X-bonds are analogous to and, thus, interchangeable with hydrogen bonds (H-bonds) and are seen to primarily replace the functions of H-bonds in recently engineered chemical catalysts.

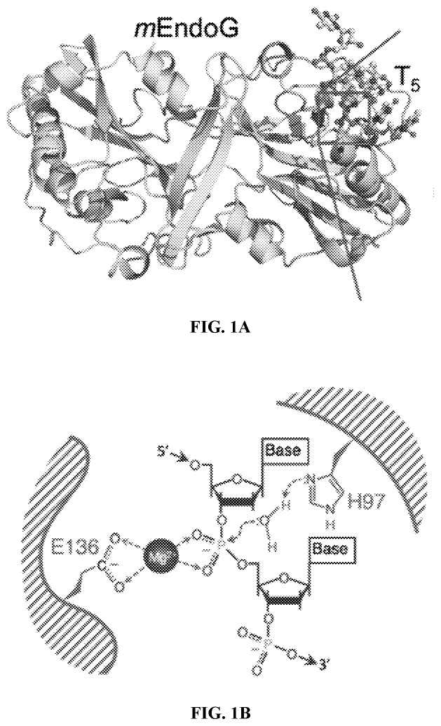

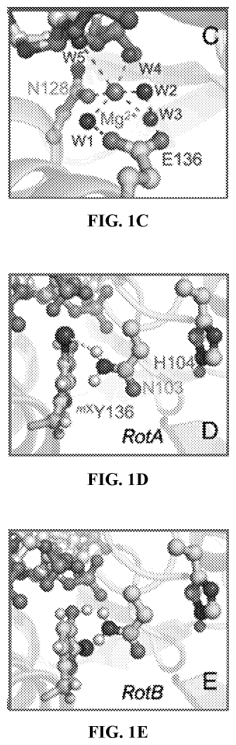

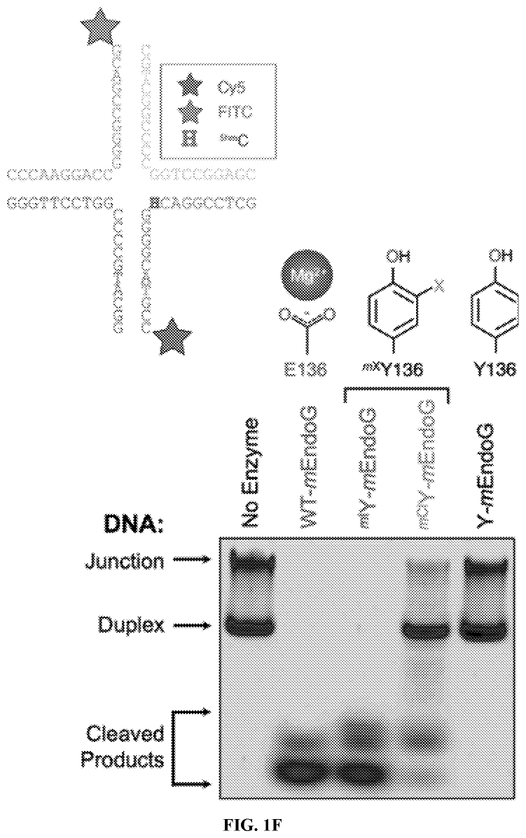

mEndoG specifically cleaves 5-hydroxymethylcytosine (^{5hm}C) modified DNAs in the context of four-stranded DNA junctions. DNA cleavage by mEndoG follows a classic endonuclease mechanism where an Mg²⁺ cofactor coordinates to and renders the DNA phosphodiester backbone susceptible to cleavage (FIG. 1B). The metal coordinating glutamate E136 in mEndoG was mutated to a meta-halotyrosine (^{mX}Y, X=chlorine or iodine) to form a ^{mX}Y-mEndoG construct that is both acid and base catalyzed. Under basic conditions, the enzyme was inactivated by the metal chelator ethylene diamine tetraacetic acid (EDTA), indicating that the halogen substituent facilitates deprotonation of tyrosyl hydroxyl group, allowing recruitment of Mg²⁺ to restore the metal-dependent catalytic center. At low pHs, the ^{mX}Y-mEndoG was resistant to EDTA inactivation and the iodinated construct was significantly more active than the chlorinated analogue. These results implicate a hydrogen bond (H-bond) enhanced X-bond as the catalyst in the ^{mX}Y-mEndoG, with asparagine N103 serving as the H-bond donor that communicates the protonation state of histidine H104 to the halogen. This model is supported by mutation studies and electrostatic potential calculations on models for the protonated and unprotonated ^{mX}Y . . . N103 . . . . H104 system compared to the Mg²⁺ coordination complex of wildtype. Overall, ^{mX}Y-mEndoG constructs developed herein, mEndoG with a meta-halogenated tyrosine (^{mX}Y), showed the same specificity as the wild type (WT) enzyme, but the essential catalytic Mg²⁺ cofactor in the mEndoG endonuclease when functionally replaced by the unnatural halogenated ^{mX}Y amino acid, created a catalytic X-bond dependent enzyme, a cX-Zyme. The inversion of the pH profile provided evidence that ^{mX}Y-mEndoG follows a catalytic mechanism that is distinct from the wildtype, in which the halogen substituent mimics the metallic electrostatic properties of the Mg²⁺ cofactor through a hydrogen bond enhanced halogen bond (HBeXB).

Section headings as used in this section and the entire disclosure herein are merely for organizational purposes and are not intended to be limiting.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents, and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context.

For the recitation of numeric ranges herein, each intervening number therebetween with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

As used herein, the modifier “about” used in connection with a value is inclusive of the stated value and has the meaning dictated by the context (for example, it includes at least the degree of error associated with the measurement of the particular value). The modifier “about” should also be considered as disclosing the range defined by the absolute values of the two endpoints. For example, the expression “from about 2 to about 4” also discloses the range “from 2 to 4.” The term “about” may refer to ±10% of the indicated number. For example, “about 10%” may indicate a range of 9% to 11%, and “about 1” may mean from 0.9-1.1. Other meanings of “about” may be apparent from the context, such as rounding off; for example, “about 1” may also mean from 0.5 to 1.4.

The term “divalent metal ion-coordinating residue,” as used herein, refers to an amino acid residue in the parent endonuclease that directly coordinates or participates in the coordination of a divalent metal ion required for catalytic activity. Such residues are typically acidic residues (e.g., aspartate, glutamate) or polar residues (e.g., histidine, asparagine) located in or near the active site.

As used herein, “nucleic acid” or “nucleic acid sequence” refers to a polymer or oligomer of pyrimidine and/or purine bases, preferably cytosine, thymine, and uracil, and adenine and guanine, respectively. The present technology contemplates any deoxyribonucleotide, ribonucleotide, or nucleoprotein component, and any chemical variants thereof, such as methylated, hydroxymethylated, or glycosylated forms of these bases, and the like. The polymers or oligomers may be heterogenous or homogenous in composition and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states. In some embodiments, a nucleic acid or nucleic acid sequence comprises other kinds of nucleic acid structures such as, for instance, a DNA/RNA helix, peptide nucleic acid (PNA), morpholino nucleic acid (see, e.g., Braasch and Corey, Biochemistry, 41(14): 4503-4510 (2002) and U.S. Pat. No. 5,034,506), locked nucleic acid (LNA; see Wahlestedt et al., Proc. Natl. Acad. Sci. U.S.A., 97:5633-5638 (2000)), cyclohexenyl nucleic acids (see Wang, J. Am. Chem. Soc., 122:8595-8602 (2000)), and/or a ribozyme. Hence, the term “nucleic acid” or “nucleic acid sequence” may also encompass a chain comprising non-natural nucleotides, modified nucleotides, and/or non-nucleotide building blocks that can exhibit the same function as natural nucleotides (e.g., “nucleotide analogs”); further, the term “nucleic acid sequence” as used herein refers to an oligonucleotide, nucleotide or polynucleotide, and fragments or portions thereof, and to DNA or RNA of genomic or synthetic origin, which may be single or double-stranded, and represent the sense or antisense strand. The terms “nucleic acid,” “polynucleotide,” “nucleotide sequence,” and “oligonucleotide” are used interchangeably. They refer to a polymeric form of nucleotides of any length, either deoxyribonucleotides or ribonucleotides, or analogs thereof.

A “peptide,” “polypeptide,” or protein is a linked sequence of two or more amino acids linked by peptide bonds. The peptide or polypeptide may be modified by the addition of sugars, lipids or other moieties not included in the amino acid chain. The terms “polypeptide,” “oligopeptide,” “protein,” and “peptide” are used interchangeably herein. The peptide may be produced by recombinant genetic technology or chemical synthesis. The peptide may be isolated and purified by any number of standard methods including, but not limited to, differential solubility (e.g., precipitation), centrifugation, chromatography (e.g., affinity, ion exchange, and size exclusion), or by any other standard techniques known in the art.

The term “amino acid” or “any amino acid” as used here refers to any and all amino acids, including naturally occurring amino acids (e.g., α-amino acids), unnatural amino acids, modified amino acids, and non-natural amino acids. It includes both D- and L-amino acids. Natural amino acids include those found in nature, such as, e.g., the 23 amino acids that combine into peptide chains to form the building blocks of a vast array of proteins. These are primarily L stereoisomers, although a few D-amino acids occur in bacterial envelopes and some antibiotics. For the most part, the names of naturally occurring and non-naturally occurring aminoacyl residues used herein follow the naming conventions suggested by the IUPAC Commission on the Nomenclature of Organic Chemistry and the IUPAC-IUB Commission on Biochemical Nomenclature as set out in “Nomenclature of α-Amino Acids (Recommendations, 1974)” Biochemistry, 14 (2), (1975). To the extent that the names and abbreviations of amino acids and aminoacyl residues employed in this specification and appended claims differ from those suggestions, they will be made clear to the reader. Throughout the present specification, unless naturally occurring amino acids are referred to by their full name (e.g., alanine, arginine, etc.), they are designated by their conventional three-letter or single-letter abbreviations (e.g., Ala or A for alanine, Arg or R for arginine, etc.). The term “L-amino acid,” as used herein, refers to the “L” isomeric form of a peptide, and conversely the term “D-amino acid” refers to the “D” isomeric form of a peptide (e.g., Dphe, (D)Phe, D-Phe, or ^{D}F for the D isomeric form of Phenylalanine). Amino acid residues in the D isomeric form can be substituted for any L-amino acid residue, as long as the desired function is retained by the peptide.

The term “halogen bond,” as used herein, refers to a primarily electrostatic, noncovalent interaction between the electrophilic region (σ-hole) of a covalently bonded halogen atom and a nucleophilic region of another molecule, such as a lone pair of electrons on an oxygen or nitrogen atom, or the π-electron density of an aromatic ring or nucleic acid base. In the context of the present disclosure, the halogen bond is formed between the halogen atom of the halogenated unnatural amino acid and a nucleophilic moiety on the target nucleic acid (e.g., a phosphodiester oxygen, a nucleobase heteroatom, or a ribose/deoxyribose oxygen).

The term “parent,” as used herein in reference to a particular protein (e.g., an endonuclease), refers to the naturally occurring or reference protein from which a modified protein is derived. In the context of the present disclosure, the parent endonuclease possesses at least one divalent metal ion-coordinating residue in or near its active site. For example, a parent endonuclease is any endonuclease that coordinates one or more divalent metal ions (e.g., Mg²⁺, Mn²⁺, Zn²⁺) at its active site through amino acid residues such as aspartate, glutamate, histidine, or asparagine.

The term “reaction medium,” as used herein, refers to the environment in which the modified endonuclease and the target nucleic acid are contacted and in which catalytic cleavage occurs. The reaction medium may be in vitro, e.g., an aqueous solution or buffer. The reaction medium may include a buffering agent capable of maintaining a desired pH, water, and salts as appropriate.

The term “substantially free of divalent metal cofactor” means that the concentration of divalent metal cations in the reaction medium is below a level sufficient to support catalytic activity of the parent endonuclease. In certain embodiments, this means the concentration of divalent metal cations is less than about 10 μM, less than about 1 μM, or less than about 0.1 μM. In certain embodiments, the reaction medium may further comprise a metal chelator to sequester trace amounts of divalent metal ions.

Preferred methods and materials are described below, although methods and materials similar or equivalent to those described herein can be used in practice or testing of the present disclosure. All publications, patent applications, patents, and other references mentioned herein are incorporated by reference in their entirety. The materials, methods, and examples disclosed herein are illustrative only and not intended to be limiting.

### 2. Modified Nucleases

Embodiments of the present disclosure include methods for utilizing modified or engineered nucleases (e.g., endonuclease). The modified nucleases are derived from parent nucleases by substitution of a halogenated amino acid at a position corresponding to a divalent metal ion-coordinating residue. In the parent nuclease, the divalent metal ion-coordinating residue participates in the binding and/or positioning of a catalytically essential divalent metal ion (e.g., Mg²⁺). By replacing this residue with a halogenated amino acid, the modified nuclease acquires the ability to form a halogen bond with the target nucleic acid at or near the site where the metal ion would otherwise interact with the target nucleic acid. Accordingly, the modified nuclease retains the ability to cleave a target nucleic acid but may differ from the parent nuclease in its catalytic mechanism, cofactor requirements, pH activity profile, or any combination thereof.

In some embodiments, the modified nuclease exhibits a catalytic mechanism that is distinct from that of the parent nuclease. In particular, the modified nuclease may operate through two distinct mechanistic modes depending on the pH of the reaction medium. At alkaline pH (e.g., above about 8.0), the halogen substituent of the halogenated amino acid may facilitate deprotonation of a hydroxyl group (e.g., a tyrosyl hydroxyl), thereby enabling recruitment of a divalent metal ion (e.g., Mg²⁺) to the active site and recapitulating a metal-dependent catalytic mechanism. At acidic pH (e.g., below about 7.0), the modified nuclease may operate through a metal-independent mechanism in which the halogen atom of the halogenated amino acid forms a halogen bond (X-bond) with the target nucleic acid, functionally mimicking the electrostatic role of the divalent metal ion cofactor. Without being bound by theory, under acidic conditions, the σ-hole of the halogen substituent is enhanced by a hydrogen-bond network involving neighboring residues, thereby increasing the electropositivity of the halogen and strengthening the halogen bond with nucleophilic moieties (e.g., phosphodiester oxygens) of the target nucleic acid.

In some embodiments, the modified nuclease exhibits a biphasic or U-shaped pH-activity profile. In such embodiments, the modified nuclease has a first activity maximum at a pH below about 7.0 and a second activity maximum at a pH above about 8.0, with a minimum of catalytic activity at a pH of about 7.0 to about 8.0. In some embodiments, the modified nuclease exhibits a pH-activity profile which is inverted relative to that of the parent nuclease. For example, in some embodiments, the parent nuclease typically exhibits maximum catalytic activity at or near neutral pH (e.g., between about pH 7.0 and about pH 7.5). The inverted pH-activity profile of the modified nuclease facilitates pH-switchable control of nuclease activity.

In some embodiments, the modified nuclease further comprises one or more amino acid residues that form a hydrogen-bond (H-bond) network with the halogenated amino acid, the H-bond network enhancing the electropositivity of the σ-hole of the halogen substituent. In such embodiments, the H-bond network communicates the protonation state of a pH-sensitive residue (e.g., a histidine) to the halogen through one or more intervening residues (e.g., an asparagine serving as an H-bond bridge). Protonation of the pH-sensitive residue under acidic conditions may create an enhanced hydrogen-bond network (e.g., a protonated histidine donating an H-bond to an asparagine, which in turn donates an H-bond to the halogen), thereby significantly increasing the electropositive character of the halogen's σ-hole and strengthening the catalytic halogen bond with the target nucleic acid. This hydrogen bond enhanced halogen bond (HBeXB) interaction functionally mimics the electrostatic properties of the divalent metal ion cofactor of the parent nuclease. In some embodiments, the H-bond network increases the electrostatic potential (ESP) at the σ-hole by about 2-fold to about 3-fold relative to the corresponding unprotonated network.

In some embodiments, the parent nuclease is endonuclease G (EndoG) or a homolog or ortholog thereof. EndoG is a mitochondrial nuclease that plays a role in apoptotic DNA degradation, mitochondrial DNA replication, and other processes. Mature EndoG is a relatively small, monomeric enzyme that cleaves both single-stranded and double-stranded DNA in a divalent metal ion-dependent manner. Homologs and orthologs of EndoG include, without limitation, EndoG proteins from mammalian species (e.g., human, mouse, rat, bovine), non-mammalian vertebrates, invertebrates (e.g., Caenorhabditis elegans CPS-6), and other eukaryotic organisms, as well as prokaryotic nucleases that share structural and/or functional homology with EndoG. Homologs and orthologs of EndoG suitable for use in the present invention include, without limitation, human EndoG, mouse EndoG, C. elegans CPS-6, Drosophila melanogaster EndoG, and EndoG orthologs from other eukaryotic species. Prokaryotic nucleases that share structural homology with EndoG, particularly at the active site metal-binding region, are also encompassed.

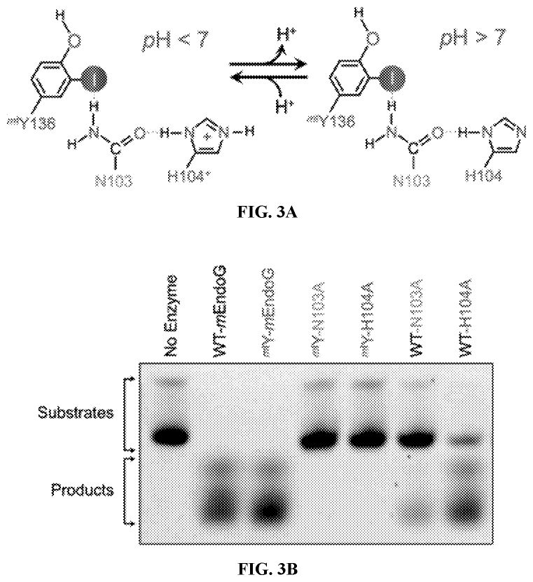

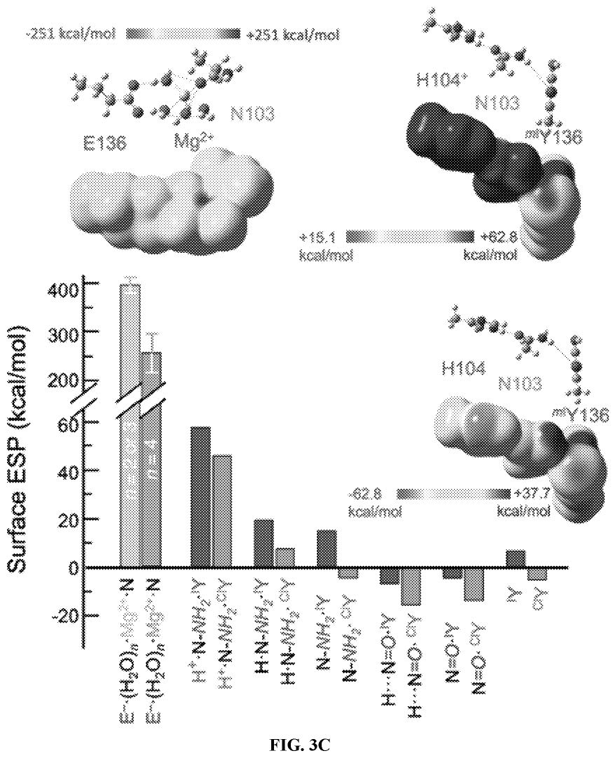

In some embodiments where the parent endonuclease is mouse EndoG (mEndoG), the divalent metal ion-coordinating residue is glutamate at position 136 (E136), and the halogenated amino acid is substituted at position 136. In the wild-type mEndoG active site, E136 coordinates the catalytic Mg²⁺ through bridging water molecules (FIG. 1C). In some embodiments, the modified endonuclease retains asparagine at position 103 (N103) and histidine at position 104 (H104), which together with the halogenated amino acid at position 136 form the H-bond network (H104 . . . N103 . . . ^{mx}Y136) described herein (FIG. 3A).

The halogenated amino acid may be incorporated into the parent endonuclease at the position corresponding to a divalent metal ion-coordinating residue using any suitable method known in the art. Non-limiting methods for site-specific incorporation of halogenated amino acid and/or unnatural amino acids include amber suppression (stop codon suppression), expanded genetic code systems, cell-free (in vitro) translation, chemical modification or semi-synthesis, and solid-phase peptide synthesis.

In amber suppression, an amber (TAG) stop codon is introduced at the desired position in the gene encoding the parent nuclease by site-directed mutagenesis. The gene is then expressed in a host cell (e.g., Escherichia coli, yeast, insect cells, or mammalian cells) that has been engineered to express an orthogonal aminoacyl-tRNA synthetase/tRNA pair capable of charging the suppressor tRNA with the desired halogenated amino acid. The halogenated amino acid (e.g., meta-iodotyrosine) is supplied in the growth medium of the host cell. Read-through of the amber codon results in site-specific incorporation of the halogenated unnatural amino acid at the designated position.

Alternatively, the modified endonuclease may be produced using: cell-free (in vitro) translation systems supplemented with the halogenated unnatural amino acid and an appropriate orthogonal aminoacyl-tRNA synthetase/tRNA pair; or expanded genetic code systems or other codon reassignment strategies, including the use of four-base codons, unnatural base pairs, or sense codon reassignment, may also be employed to incorporate the halogenated unnatural amino acid.

The halogenated amino acid may also be introduced by chemical modification of a recombinant protein, expressed protein ligation, native chemical ligation, or other semi-synthetic approaches. For smaller nucleases or fragments thereof, the modified nuclease may be produced entirely by chemical synthesis.

Halogenated amino acids are amino acids that bear at least one halogen substituent (e.g., elements from group VII of the periodic table, including fluorine, chlorine, bromine, and iodine) on its side chain. The halogenated amino acids may be modified, engineered or unnatural analogs of one of the twenty canonical amino acids encoded by the standard genetic code. As described above, however, the halogenated amino acids are not capable of being incorporated into a growing polypeptide chain using normal transcription/translation machinery, including canonical aminoacyl-tRNA synthetase/tRNA pairs.

In some embodiments, the halogenated amino acid is a halogenated tyrosine. A halogenated tyrosine is a tyrosine analog in which one or more hydrogen atom substituents on the phenyl ring of the tyrosine side chain have been replaced by a halogen atom. In certain embodiments, the halogenated tyrosine is a meta-halotyrosine having a halogen substituent at the meta position (3-position) of the phenyl ring. Exemplary halogenated tyrosine analogs include, but are not limited to, 3-fluoro-L-tyrosine, 3-chloro-L-tyrosine, 3-bromo-L-tyrosine, and 3-iodo-L-tyrosine (meta-fluorotyrosine, meta-chlorotyrosine, meta-bromotyrosine, and meta-iodotyrosine, respectively). In select embodiments, the halogenated amino acid is meta-iodotyrosine.

The modified nucleases are not limited by the halogenated amino acid. However, the position and geometry of the halogen atom relative to the active site and the target nucleic acid are important for catalytic function. The halogenated amino acid may also be a halogenated phenylalanine, a halogenated tryptophan, a halogenated histidine, or another halogenated amino acid that is capable of forming a halogen bond with a nucleophilic moiety on the target nucleic acid (e.g., a phosphodiester oxygen, a nucleobase heteroatom, or a ribose/deoxyribose oxygen).

The disclosure also provides for modified nucleases (e.g., modified endonucleases) described herein and compositions and kits comprising the modified nucleases (e.g., modified endonucleases). The compositions may further comprise a buffering agent, a monovalent salt, a reducing agent, a nonionic detergent, a stabilizer, or any combination thereof. The kits may further comprise one or more reaction buffers or components thereof (e.g., a buffering agent, a monovalent salt, a reducing agent,). The compositions and kits may optionally further comprise a metal chelator (e.g., EDTA), a divalent metal cofactor salt (e.g., MgCl₂), a target nucleic acid, a control nuclease and control nucleic acid substrate, and/or instructions for use.

### 3. Methods

In one aspect the disclosure provides methods of modulating the catalytic activity of a modified nuclease. In some embodiments, the methods comprise: contacting the modified endonuclease with a target nucleic acid and adjusting the pH to activate cleavage.

As described above, the modified endonuclease comprises a halogenated amino acid substituted at a position corresponding to a divalent metal ion-coordinating residue of a parent endonuclease. The halogenated amino acid forms a halogen bond with a target nucleic acid. Any modified endonuclease as described above is suitable for use in the disclosed methods. In some embodiments, the halogenated amino acid is a halogenated tyrosine. In select embodiments, the halogenated amino acid is a meta-halotyrosine. In exemplary embodiments, the halogenated amino acid is meta-iodotyrosine. In some embodiments, the parent endonuclease is EndoG or a homolog or ortholog thereof.

The modified endonuclease may be utilized in purified form, from a crude cell lysate, or in any other suitable form. In certain embodiments, the modified endonuclease is in a buffered solution at a storage pH that maintains stability of the enzyme.

The modified endonuclease is contacted with the target nucleic acid. The target nucleic acid is not limited by sequence, structure, modification, size, or any other characteristic, on condition that it is recognized and cleaved by the modified endonuclease. In some embodiments, the target nucleic acid is DNA, including but not limited to double-stranded DNA (dsDNA), single-stranded DNA (ssDNA), plasmid DNA, linear DNA, and genomic DNA. In some embodiments, the target nucleic acid is RNA or a DNA-RNA hybrid.

In some embodiments, the target nucleic acid comprises one or more modified nucleobases, such as a modified cytosine. In certain embodiments, the modified cytosine is 5-hydroxymethylcytosine (^{5hm}C). In some embodiments, the target nucleic acid is in the form of a multi-stranded nucleic acid structure, such as a three-stranded junction, a four-stranded junction (e.g., a Holliday junction or a four-way DNA junction), or another branched nucleic acid structure. In exemplary embodiments, the target nucleic acid is a four-stranded DNA junction comprising at least one ^{5hm}C-modified strand. In some embodiments, the modified endonuclease preferentially cleaves multi-stranded junction substrates over linear duplex substrates.

In some embodiments, the target nucleic acid and modified endonuclease are contacted in a reaction medium. In some embodiments, the reaction medium is an aqueous solution comprising a buffering agent and, optionally, salts, reducing agents, stabilizers, and other additives as appropriate for the particular application.

The reaction medium is adjusted to a pH where the modified endonuclease cleaves the target nucleic acid. In certain embodiments, this activation pH is below about 7.0 (e.g., about 7.0, about 6.9, about 6.8, about 6.7, about 6.6, about 6.5, about 6.4, about 6.3, about 6.2, about 6.1, about 6.0, about 5.9, about 5.8, about 5.7, about 5.6, about 5.5, or lower) or above about 8.0 (e.g., about 8.0, about 8.1, about 8.2, about 8.3, about 8.4, about 8.5, about 8.6, about 8.7, about 8.8, about 8.9, about 9.0, about 9.1, about 9.2, about 9.3, about 9.4, about 9.5 or higher). In further embodiments, the activation pH is from about 5.5 to about 7.0 (acidic activation) or from about 8.0 to about 10.0 (alkaline activation).

Without being bound by theory, at pH values below about 7.0 (acidic conditions), the protonation states of the halogenated tyrosine and/or the target nucleic acid are altered relative to neutral pH, which may strengthen or otherwise modulate the halogen bond between the halogenated amino acid and the nucleic acid, thereby promoting catalysis. Similarly, at pH values above about 8.0 (alkaline conditions), deprotonation of the phenolic hydroxyl of the halogenated tyrosine and/or alterations in nucleic acid protonation may enhance halogen bonding and catalytic activity.

The pH of the reaction medium may be adjusted by any suitable means, including but not limited to the addition of acid (e.g., HCl, acetic acid) or base (e.g., NaOH, Tris base), dilution with a buffer of the desired pH, dialysis, or electrochemical methods.

In some embodiments, the method further comprises adjusting the pH of the reaction medium to a pH that reduces the catalytic activity of the modified endonuclease. In select embodiments, this “off” pH is about 7.0 to about 8.0 (e.g., approximately physiological or neutral pH). Thus, the modified endonuclease exhibits a unique biphasic or U-shaped pH-activity profile, with high activity at acidic pH (below about 7.0) and/or at alkaline pH (above about 8.0), and reduced activity at neutral pH (about 7.0 to about 8.0).

This pH-switchable behavior enables the sequential and reversible activation and deactivation of the modified endonuclease by alternating the pH of the reaction medium between a pH at which the endonuclease is active (e.g., below about 7.0 or above about 8.0) and a pH at which the endonuclease is less active or inactive (e.g., between about 7.0 and about 8.0). In some embodiments, the method comprises sequentially adjusting the pH of the reaction medium one or more times between the pH at which the endonuclease is active and the pH at which the endonuclease is less active or inactive. This facilitates temporal control over nuclease activity, enabling applications such as controlled, stepwise digestion of nucleic acids; timed cleavage events in multi-step molecular biology workflows; and regulated nuclease activity in diagnostic or therapeutic settings.

In another aspect the disclosure provides methods for cleaving a target nucleic acid using a modified endonuclease. The methods comprise providing a modified endonuclease, as described herein, and contacting it with a target nucleic acid at a selected pH where the modified endonuclease cleaves the target nucleic acid.

In some embodiments, the select pH is below about 7.0 or above about 8.0. In some embodiments the select pH is from about 5.5 to about 7.0 or from about 8.0 to about 10.0. The modified endonuclease may exhibit reduced catalytic activity at a pH of about 7.0 to about 8.0 relative to its activity at a pH below about 7.0 or above about 8.0, consistent with the pH-switchable behavior described above. In some embodiments, the method may further comprise alternating the pH between a pH at which the endonuclease is active (e.g., below about 7.0 or above about 8.0) and a pH at which the endonuclease is less active or inactive (e.g., between about 7.0 and about 8.0).

### a) Reaction Conditions

In some embodiments, where the pH (e.g., the select pH or pH of the reaction medium) is below about 7.0, the method does not comprise contacting the modified endonuclease and/or target nucleic acid with a divalent metal cofactor and/or the reaction medium is substantially free of divalent metal cofactor. Divalent metal cofactors include any divalent metal cation (e.g., Mg²⁺, Mn²⁺, Ca²⁺, Zn²⁺). The divalent metal cofactors include those cofactors utilized by the parent nuclease for catalytic activity. The ability to preserve nuclease activity without requiring divalent metal cofactors is a significant advantage over the parent endonuclease. The ability of the modified endonuclease to cleave target nucleic acids in the absence of divalent metal cofactors simplifies reaction formulations, eliminates the need for exogenous metal ion supplementation, and enables nuclease activity under conditions (e.g., in the presence of metal chelators) where the parent endonuclease would be inactive.

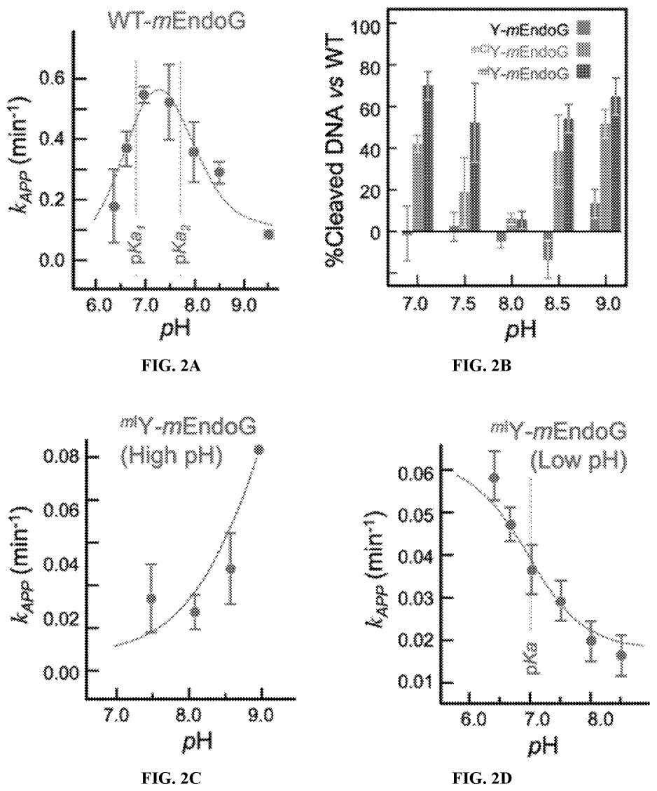

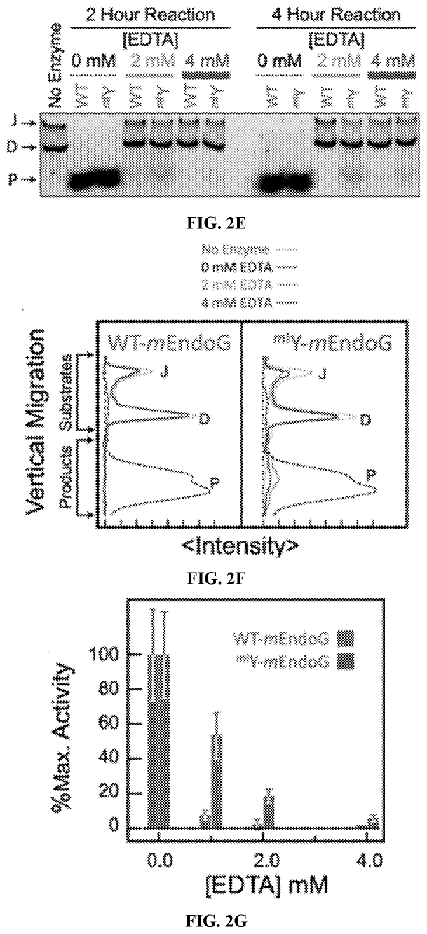

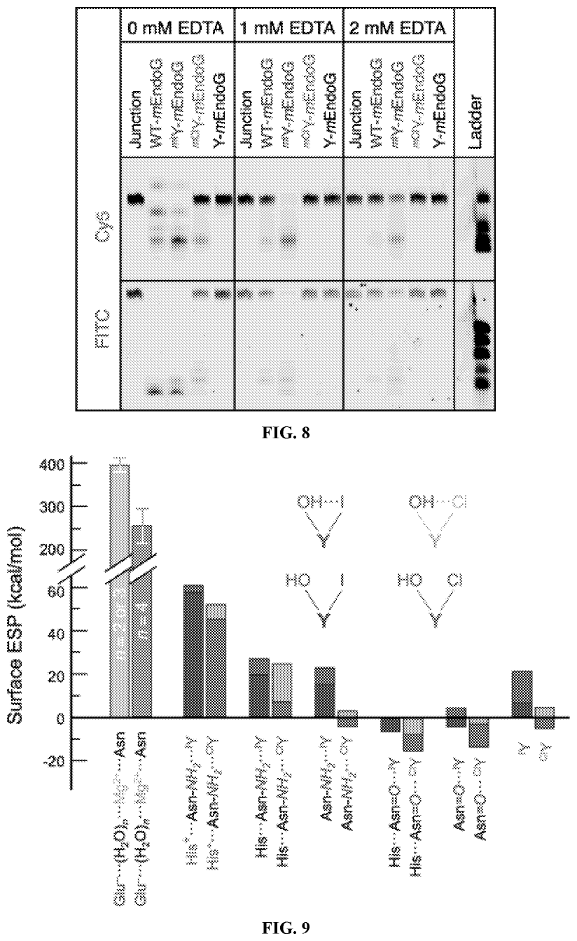

In some embodiments, the modified endonuclease retains catalytic activity in the presence of a metal chelator at a concentration sufficient to substantially inactivate the parent endonuclease. Metal chelators are compounds that bind divalent metal ions with high affinity, thereby reducing the concentration of free divalent metal ions in solution. Non-limiting examples include ethylenediaminetetraacetic acid (EDTA), ethylene glycol-bis(β-aminoethyl ether)-N,N,N′,N′-tetraacetic acid (EGTA), and 1,2-bis(o-aminophenoxy) ethane-N,N,N′,N′-tetraacetic acid (BAPTA). In exemplary embodiments, at acidic pH (e.g., pH 7.0 or below), the modified endonuclease retains significant catalytic activity in the presence of up to about 2 mM or up to about 4 mM EDTA, whereas the parent endonuclease loses greater than about 90% of its catalytic activity at about 1 mM EDTA under otherwise identical conditions (FIGS. 2E-2G, FIG. 8).

The phrase “at a concentration sufficient to substantially inactivate the parent” means a concentration of metal chelator that reduces the catalytic activity of the parent to less than 50% (e.g., to less than about 40%, to less than about 30%, to less than about 20%, to less than about 10%, less than about 5%, or less than about 1%) of the activity observed in the absence of the chelator, under otherwise identical conditions. For example, the modified endonuclease may retain at least about 50%, at least about 60%, at least about 70%, at least about 80%, or at least about 90% of its catalytic activity in the presence of a chelator. In some embodiments, the modified endonuclease may retain at least about 50% of its catalytic activity in the presence of 1 mM EDTA, 5 mM EDTA, 10 mM EDTA, or higher concentrations, whereas the parent endonuclease is substantially inactive under the same conditions.

In embodiments where the pH is above about 8.0, the method may further include contacting the modified endonuclease and/or target nucleic acid with a divalent metal cofactor and/or the reaction medium may include a divalent metal cofactor. Without being bound by theory, in such embodiments, the modified endonuclease may utilize a synergistic catalytic mechanism in which both the halogen bond and the divalent metal ion contribute to substrate binding and/or transition state stabilization. The concentration of divalent metal cofactor in the reaction medium may be, for example, from about 0.1 mM to about 50 mM, from about 1 mM to about 20 mM, or from about 5 mM to about 10 mM. In some embodiments, the divalent metal cation is Mg²⁺.

In some embodiments, the reaction medium may further comprise one or more of the following components: a monovalent salt; a buffering agent; a reducing agent; a nonionic detergent; enzyme stabilizer and/or blocking agent.

The concentration and identity of the monovalent salt may be selected to maintain ionic strength, promote enzyme-substrate interaction, and/or stabilize nucleic acid secondary structures (e.g., multi-stranded junctions) under the chosen reaction conditions. Non-limiting exemplary monovalent salts include sodium chloride (NaCl), potassium chloride (KCl), sodium acetate (NaOAc), potassium acetate (KOAc), ammonium chloride (NH₄Cl), and combinations thereof. In some embodiments, the monovalent salt is at a concentration of from about 1 mM to about 1 M, from about 10 mM to about 500 mM, from about 25 mM to about 250 mM, from about 50 mM to about 150 mM, or about 50 mM.

In certain embodiments, the monovalent salt is NaCl at a concentration of about 50 mM. In some embodiments, the reaction medium comprises NaCl at a concentration of from about 10 mM to about 200 mM and KCl at a concentration of from about 10 mM to about 200 mM, individually or in combination.

Non-limiting exemplary agents include buffering tris(hydroxymethyl)aminomethane (Tris), 4-(2-hydroxyethyl)-1-piperazineethanesulfonic acid (HEPES), 2-(N-morpholino) ethanesulfonic acid (MES), 3-(N-morpholino) propanesulfonic acid (MOPS), piperazine-N,N′-bis(2-ethanesulfonic acid) (PIPES), acetate, phosphate, citrate, bis-tris propane, and combinations thereof. The buffering agent may be selected to have an effective buffering range (pKa) appropriate for the desired reaction pH. For example, MES (pKa≈6.1) or acetate (pKa≈4.75) may be preferred for reactions conducted under acidic conditions (e.g., below about pH 7.0); Tris (pKa≈8.1), HEPES (pKa≈7.5), or MOPS (pKa≈7.2) may be preferred for reactions conducted at neutral to mildly alkaline pH; and bis-tris propane (pKa≈6.8 and 9.0) or a Tris-based buffer may be preferred for reactions conducted under alkaline conditions (e.g., above about pH 8.0). In embodiments where pH-switching is employed, the reaction medium may comprise a combination of two or more buffering agents to provide effective buffering capacity across the range of pH values used (e.g., from about pH 5.5 to about pH 10.0).

In some embodiments, the buffering agent is at a concentration of from about 1 mM to about 500 mM, from about 5 mM to about 200 mM, from about 10 mM to about 100 mM, from about 15 mM to about 50 mM, or about 20 mM. In certain embodiments, the buffering agent is Tris at a concentration of about 20 mM. In some embodiments, the buffering agent is Tris at about pH 7.5 and at a concentration of about 20 mM.

The reducing agent may be included to maintain the modified endonuclease in a reduced state and to prevent oxidation of cysteine residues or other oxidation-sensitive moieties. Exemplary reducing agents include, but are not limited to, dithiothreitol (DTT), β-mercaptoethanol (BME), tris(2-carboxyethyl)phosphine (TCEP), glutathione, and cysteine. In some embodiments, the reducing agent is at a concentration of from about 0.01 mM to about 50 mM, from about 0.1 mM to about 10 mM, from about 1 mM to about 20 mM, from about 5 mM to about 15 mM, or about 10 mM. In certain embodiments, the reducing agent is BME at a concentration of about 10 mM. In some embodiments, the reducing agent is DTT at a concentration of from about 0.5 mM to about 5 mM, or about 1 mM.

The nonionic detergent may be included to prevent nonspecific adsorption of reaction components to vessel surfaces, to reduce aggregation, and/or to improve the reproducibility of the reaction. Non-limiting exemplary nonionic detergents include Triton X-100, Tween-20, Nonidet P-40, nonyl phenoxypolyethoxylethanol, and octyl glucoside. In some embodiments, the nonionic detergent is at a concentration of from about 0.001% to about 2% (v/v), from about 0.01% to about 1% (v/v), from about 0.05% to about 0.5% (v/v), or about 0.1% (v/v). In certain embodiments, the nonionic detergent is Triton X-100 at a concentration of about 0.1% (v/v).

Enzyme stabilizers and/or blocking agents, such as bovine serum albumin (BSA), gelatin, and glycerol may also be included in the reaction medium. Enzyme stabilizers and/or blocking agents can saturate surfaces, preventing the enzyme from sticking and becoming inactivated, and maintain enzyme conformation by providing a protective protein environment or stabilizing active conformations. BSA can also reduce nonspecific binding between the enzyme and non-target molecules that may be in the reaction medium, thereby improving the specificity and reproducibility of the reaction. The concentration may depend on the specific stabilizer or blocking agent. For example, BSA and/or gelatin may be at a concentration of from about 0.01 mg/mL to about 1 mg/mL. Glycerol may be at a concentration of from about 1% to about 20% (v/v).

The reaction temperature depends on the nuclease being used. For example, the reaction temperature may be from about 4° C. to about 65° C., from about 20° C. to about 42° C., and from about 25° C. to about 37° C., unless a thermostable variant of the modified endonuclease is employed. Generally, the reaction temperature known for the parent nuclease will be suitable for use with the modified nuclease.

The concentration of the modified endonuclease depends on the application, the amount and form of target nucleic acid to be cleaved, the desired degree and rate of cleavage, and the specific activity of the modified endonuclease under the chosen pH conditions. The concentration of the modified endonuclease in the reaction medium may be from about 0.01 nM to about 100 μM, from about 0.1 nM to about 10 μM, from about 1 nM to about 1 μM, from about 5 nM to about 500 nM, from about 10 nM to about 100 nM, or from about 50 nM to about 500 nM. In some embodiments, the concentration of the modified endonuclease is from about 0.1 μM to about 5 μM, from about 0.25 μM to about 2 μM, or about 0.5 μM.

In some embodiments, the modified endonuclease is provided at a molar excess relative to the target nucleic acid. In some embodiments, the molar ratio of modified endonuclease to target nucleic acid is from about 2:1 to about 100:1, from about 5:1 to about 50:1, or from about 10:1 to about 20:1.

The concentration of target nucleic acid may be selected depending on the application and the sensitivity of the detection or downstream processing method employed. The concentration of target nucleic acid in the reaction medium may be from about 0.1 ng/μL to about 10 μg/μL, from about 0.5 ng/μL to about 5 μg/μL, from about 1 ng/μL to about 1 μg/μL, from about 5 ng/μL to about 500 ng/μL, or from about 10 ng/μL to about 100 ng/μL. In some embodiments, the concentration of the target nucleic acid is from about 1 nM to about 10 μM, from about 5 nM to about 1 μM, from about 10 nM to about 500 nM, from about 20 nM to about 200 nM, or about 45 nM to about 50 nM.

The reaction time depends on the enzyme concentration, substrate concentration, pH, temperature, desired degree of cleavage, and whether partial or complete digestion of the target nucleic acid is desired. The reaction time may be from about 30 seconds to about 48 hours, from about 1 minute to about 24 hours, from about 5 minutes to about 12 hours, from about 5 minutes to about 4 hours, from about 10 minutes to about 2 hours, from about 15 minutes to about 90 minutes, or from about 20 minutes to about 60 minutes. In some embodiments, the reaction time is from about 1 hour to about 8 hours, from about 2 hours to about 6 hours, or about 4 hours. In select embodiments, the reaction time is about 5 minutes, about 10 minutes, about 20 minutes, about 30 minutes, about 60 minutes, about 90 minutes, about 2 hours, or about 4 hours.

### b) Applications

The methods of the present invention find utility in a wide range of applications.

The methods may be suitable for use in controlled nucleic acid digestion. The pH-switchable activity of the modified endonuclease enables precise temporal control over nucleic acid cleavage, which is useful in applications such as library preparation for next-generation sequencing, fragmentation of genomic DNA, and controlled degradation of unwanted nucleic acids in sample preparation workflows.

The methods may be suitable for use in nucleic acid sequencing. The ability to activate and deactivate nuclease activity by pH adjustment may be exploited in sequencing-by-synthesis or other sequencing methodologies that benefit from controlled, stepwise nucleic acid cleavage.

The methods may be suitable for use in molecular diagnostics. The modified endonuclease may be used as a signal-generating or signal-amplifying component in diagnostic assays, where pH-triggered cleavage of a reporter nucleic acid substrate produces a detectable signal.

The methods may be suitable for use in therapeutic applications. The pH-responsive activity of the modified endonuclease may be exploited in therapeutic contexts where localized activation is desired, e.g., in the acidic microenvironment of tumors, endosomes, or lysosomes.

The methods may be suitable for use in metal-free nuclease reactions. The ability of the modified endonuclease to cleave DNA at acidic pH in the substantial absence of divalent metal cofactors is advantageous in applications where metal ions are undesirable, such as in certain analytical techniques (e.g., mass spectrometry-based workflows) or in environments where metal chelators are present.

The methods may be suitable for use in enzyme engineering and structure-function studies. The modified endonucleases of the present invention serve as tools for investigating the role of halogen bonding in enzymatic catalysis and for developing novel enzyme designs based on non-canonical interactions.

### 4. Examples

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to herein are hereby incorporated by reference in their entireties.

The present disclosure has multiple aspects, illustrated by the following non-limiting examples.

### Example 1

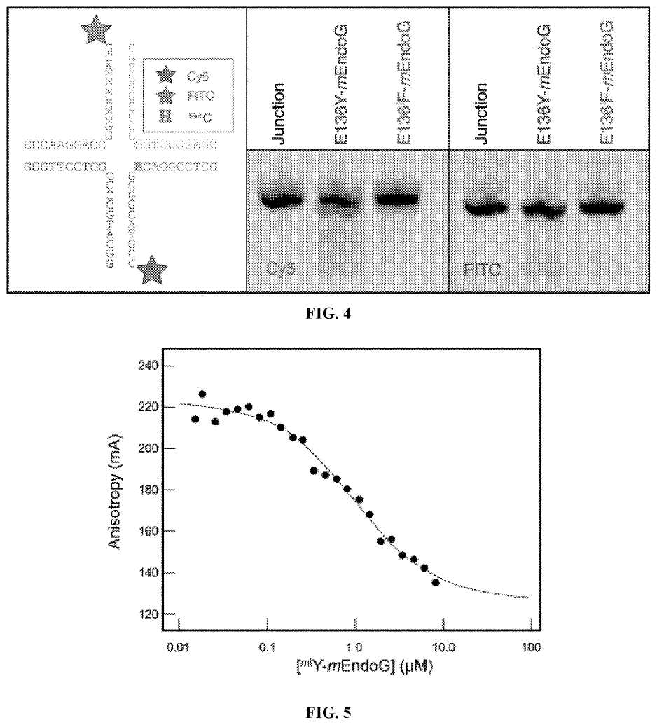

Replacing E136 (FIG. 1C) in mEndoG with an ^{mX}Y (X=Cl or I, FIGS. 1D-1E) creates ^{mX}Y-mEndoG constructs that cleave ^{5hm}C-modified DNA junction and duplex substrates (FIG. 1F), resulting in products that are identical in size to those from the WT-mEndoG. In contrast, the unhalogenated tyrosyl E136Y mutant is significantly less active across all conditions, indicating that the halogen substituent of the ^{mX}Y ncAA is the essential catalytic element in the ^{mX}Y-mEndoGs. The para-phenylalanine construct (^{pX}F) is similarly or less inactive (FIG. 4), meaning that simply adding a halogen to any aromatic amino acid is insufficient to induce catalysis.

The apparent rate constants (k_{APP}) for catalytic cleavage of ^{5hm}C-junctions by WT-mEndoG under pseudo-first order reaction conditions, where the enzyme is in excess, had previously been determined to be ~0.2 to 0.5 min-1 and are reproduced here. The equivalent rate for ^{mI}Y-mEndoG (k_{APP}≈0.06 min-1) is ~10-fold lower than that of WT-mEndoG at pH 7.0, while the chlorinated ^{mCI}Y-mEndoG construct (k≈0.03 min-1) is ~2-fold lower than ^{mI}Y-mEndoG and ~20-fold lower than WT-mEndoG. The higher activity of ^{mI}Y-mEndoG over ^{mCI}Y-mEndoG mirrors the dependence of the σ-hole on the size and the polarizability of halogen substituents, thereby implicating X-bonding as responsible for facilitating DNA cleavage. The ^{mX}Y-mEndoGs are significantly more active than the Mg²⁺ dependent EndoG ortholog from Caenorhabditis elegans, which is >200-fold less active (k_{APP}≈8×10⁻⁴ min⁻¹) than WT-mEndoG. Thus, the ^{mX}Y-mEndoGs catalyzed reactions fall well within the range of rates for this class of Mg²⁺ dependent DNA nucleases. Furthermore, the ^{mX}Y-mEndoGs show the same specificity for four-stranded junctions over duplex DNA as WT-mEndoG, as evident in ^{mCI}Y-mEndoG (FIG. 1F), where nearly all the junction and very little of the duplex DNAs are cleaved.

The differences in activities among the ^{mX}Y-mEndoG constructs and relative to WT-mEndoG are not defined by their binding affinities to the ^{5hm}C-junction substrate (FIG. 5 and Table 1). While the iodinated ^{mI}Y-mEndoG shows a ~17-fold lower affinity for the substrate compared to WT, that of the chlorinated ^{mCI}Y-mEndoG for the DNA substrate is higher than both WT-mEndoG and ^{mI}Y-mEndoG, indicating that the activities of the halogenated enzyme constructs are dependent on the intrinsic catalytic potential of the halogen.

Table 1. Dissociation constants (K_{D}) of wild type (WT) and various halogenated and unhalogenated mouse endonuclease G (mEndoG) constructs with DNA Holliday junctions, as determined by fluorescence polarization assays.

| Construct | K_{D} (nM) |
|---|---|
| WT-mEndoG | 57 ± 9 |
| Y-mEndoG | 490 ± 75 |
| ^{mI}Y-mEndoG | 980 ± 191 |
| ^{mCl}Y-mEndoG | 390 ± 124 |

### Example 2

The pH dependence of ^{mI}Y-mEndoG and WT-mEndoG were assayed to probe the differences in their catalytic mechanisms. WT-mEndoG showed a pH profile (FIG. 2A) with the maximum activity seen between pH 7.0 and 7.5, associated with two pKa values (pKa₁=6.8 and pKa₂=7.7). The increase in activity going from pH 6 to 7.5 is consistent with a mechanism in which His97 acts as a general base to increase the nucleophilicity of the hydrolytic water (FIG. 1B). The reduction in activity at pH≥7.7 was interpreted as the enzyme switching from overall positively to negatively charged, thereby reducing its affinity for the negative DNA substrate. This interpretation is supported by an isoelectric point pI=8.1 calculated for mEndoG using the Isoelectric Point Calculator 2.0.

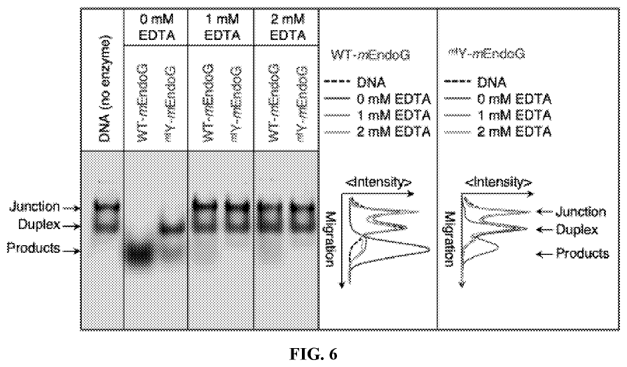

The pH profile for ^{mI}Y-mEndoG is inverted relative to that of the wildtype, showing high activities both above and below pHs 8.0 (FIG. 2B). The two pH effects were probed separately. The high pH active ^{mI}Y-mEndoG has a pKa≈8.7 (FIG. 2C) and is as or more sensitive than the wildtype to inactivation by the metal chelator ethylenediaminetetraacetic acid (EDTA, FIG. 6). These results were interpreted as the halogen lowering the pKa for deprotonation of the tyrosyl hydroxyl group through standard inductive effects (the pKa of the OH of ^{mX}Y is reported to be ~8.3 for both the chloro- and iodo-forms). This mechanism may also account for the nominal activity observed for the non-halogenated tyrosyl mutant. The sensitivity to EDTA inactivation suggests that the deprotonated ^{mX}Y recruits an Mg²⁺ to the active site, recapitulating the metal-dependent mechanism of the wildtype enzyme.

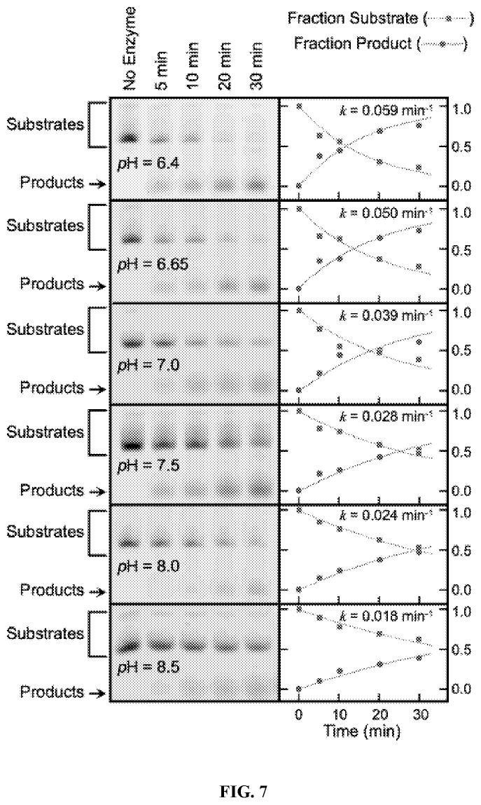

The higher activity under acidic conditions is consistent with general acid facilitated catalysis, with a single pKa value of ~6.95 (FIGS. 2D and 7). Under these conditions, ^{mI}Y-mEndoG is significantly less sensitive to EDTA inactivation than the WT-mEndoG (FIGS. 2E-2F), with the wildtype losing over 90% of its activity at 1 mM EDTA (FIG. 8) while the iodoenzyme shows residual activity up to 4 mM EDTA. Thus, the ^{mI}Y-mEndoG construct is shown to be significantly less metal-dependent than wildtype. The loss of activity of ^{mI}Y-mEndoG at higher EDTA concentrations may be attributed to the change in conformation of the junction substrate as the chelator strips Mg²⁺ from the DNA, an effect seen with other DNA junction processing enzymes.

### Example 3

Prior studies on T4 lysozyme had shown that the hydroxyl group of a ^{mX}Y amino acid can serve as the H-bond donor to an HBeXB interaction. However, the pKa≈7 for this reaction is ~1.3 pH units lower than any singly halogenated tyrosine found in the literature, thereby precluding the tyrosyl OH and pointing to a histidine being the more likely H-bond donor to the HBeXB.

In surveying the WT-mEndoG structure, H104 was found to be the closest histidine to the ^{mI}Y residue but is too distant to be a direct H-bond donor. However, an intervening asparagine (N103) is present that could accept an H-bond from H104 and donate an H-bond to ^{mI}Y (FIG. 1C). This arrangement suggested that the pH-dependent protonation state of H104 could be communicated to the halogen through the H-bonded N103 bridge (FIG. 3A). To test this model, N103 or H104 were separately mutated to alanine residues (N103A and H104A mutants, respectively) in the ^{mI}Y-mEndoG and WT-mEndoG constructs (FIG. 3B). Both the N103A and H104A mutants of ^{mI}Y-mEndoG were found to be inactive. In contrast, the N103A-WT-mEndoG showed diminished but substantial activity, while the H104A-WT-mEndoG mutant showed near wildtype activity. Thus, both N103 and H104 facilitate the catalytic activity of ^{mI}Y-mEndoG but neither are required in the catalytic mechanism of WT-mEndoG.

The electrostatic potential (ESP) surfaces of simplified molecular models that represent the various protonated and unprotonated H104 . . . N103 . . . . X networks were calculated and compared to those of models for the coordinated Mg²⁺ environment (FIG. 3C). The ESP surfaces were calculated for the ^{mX}Y136 models for both iodine and chlorine as the X substituent.

In the published crystal structure, the NH₂ of the amide side chain points towards H104, which at first would preclude it from serving as an H-bond donor to the halogen of ^{mI}Y136. However, X-ray diffraction cannot distinguish between an oxygen and nitrogen at the reported resolution and, therefore, it is not clear what the actual orientation of the amide is in respect to H104 and ^{mI}Y136 and whether this H104 . . . N103 . . . . X network model would involve rotation of N103. The ESP calculations consider both orientations of the amide with either the carbonyl O or the amino NH₂ pointing towards the halogen. However, the difference in overall energies calculated for these complexes with the carbonyl oxygen pointed towards the halogen (as in the H104 . . . N103=O . . . X model) is +132 kcal/mol (for iodine) to +138 kcal/mol (for chlorine) compared to models with the amino group towards the halogen. This large positive energy difference strongly supports the H104 . . . N103-NH₂ . . . . X model as being most likely for these studies.

The maximum ESP for Mg²⁺ in its coordination complex is highly positive (>200 kcal/mol), as expected for a dicationic metal. This complex was modeled with the Mg²⁺ coordinated to four waters, as seen in the crystal structure or 1 or 2 waters (labeled W4 and W5 in FIG. 1C) removed, leaving 2 or 3 solvent molecules coordinated to mimic the metal complex when DNA sits in the active site (there was very little difference in the calculated ESP between these two water depleted complexes). The neutral halogens in all the ^{mX}Y136 models are significantly less positive (≤60 kcal/mol). The ESP values of models calculated with the carbonyl oxygen of the N103 amide pointed towards either the ^{mX}Y136 is either negative or only slightly positive and overall less positive than that of a halophenol model alone. In contrast, all models with the amide NH₂ pointed towards the halogen have significantly positive or near neutral maximum ESP surfaces. The observation that ^{mI}Y-mEndoG shows at least weak activity under neutral pH conditions suggests that the amide is oriented to form an NH₂ . . . . IH— bond. Addition of the imidazole side chain of H104 to the model, whether protonated or not, renders all the ESPs to be positive. When this H104 imidazole is protonated, the maximum ESP values increase significantly.

The ESP of each H104 . . . N103 . . . . X model was also compared with the tyrosyl hydroxyl pointed either towards or away from the halogen to determine how much a potential OH . . . X HBeXB could contribute to the electropositive σ-hole (FIG. 9). In the models absent the protonated H104+, this OH . . . X interaction contributed significantly (upwards of 18 kcal/mol) but the overall ESP is less than that of the respective H104+ . . . N103 . . . . X models. With the H104 protonated and positively charged, the OH . . . X H-bond contributed less than 10 kcal/mol (<10%) of the electropositive charge for either the iodinated or chlorinated ESP values. Thus, above neutral pHs, the tyrosyl OH could create a significantly positive σ-hole but that this is dwarfed by the effects of the protonated H104⁺ under acidic conditions where the ^{mX}Y-mEndoG constructs are found to be most active.

Comparing the various ESP values facilitates determination of how large a role electrostatics plays in the mechanism of ^{mI}Y-mEndoG versus WT-mEndoG. The ESP of the protonated H104 . . . N103 . . . . I model network is ~2-3-fold more positive than the neutral network with the unprotonated H104, which corresponds well with the ~3-fold higher k_{APP} at pH 6.5 versus 8.5 (FIG. 2D). In addition, the iodinated enzyme shows ~60% higher activity at pH 6.5 compared to the chlorinated analogue, while their ESPs differ by ~40%. Thus, electrostatics can account generally for the acid facilitated catalysis and the difference in reactivity between the two halogens. Finally, the ESPs of the ^{mX}Y-mEndoG models can be compared to those of the Mg²⁺ coordination complex. The k_{APP} for ^{mI}Y-mEndoG at pH 6.5 is ~9-fold lower than that of WT-mEndoG, which is comparable to the ~7-fold difference in ESP values for the protonated H104H⁺. . . N103 . . . . I network and the 2/3 water coordinated complexes of Mg²⁺. Thus, the ESP calculations correlate well with the relative activities of the mEndoG constructs studied here. These findings indicate that the enhancement of the σ-hole by the H104H⁺. . . N103 . . . . I network mimics the electrostatic effects of the Mg²⁺ cofactor in the mEndoG active site and generally supports our supposition that the DNA cutting activity of the ^{mX}Y-mEndoG enzyme under acidic conditions is catalyzed by the resulting HBeXB interaction.

### Materials and Methods

Protein Constructs and Expression. Expression vectors for various forms of mouse endonuclease G (mEndoG) were constructed starting with the pET-28a plasmid containing the gene for the maltose binding protein (MBP)/mEndoG fusion protein (truncated at the N-terminus by 41 amino acids), as previously described (A. B. Robertson, et al., Nucleic Acids Res. 42, 13280-13293 (2014), C. M. Vander Zanden, et al., Nucleic Acids Res. 48, 3962-3974 (2020)). The MBP-mEndoG fusion is followed downstream by the sequence for a T7 promoter and the inhibitor dEndoGI from Drosophila melanogaster. This starting construct expresses the wildtype (WT) form of mEndoG. An inactive form of the enzyme was constructed by replacing the Mg²⁺ chelating glutamate E136 with a tyrosine (the E136Y mutant). Expression vectors for haloenzymes containing unnatural meta-halogenated tyrosine (^{mX}Y) amino acids were constructed by replacing E136 in WT-mEndoG with the AMBER stop codon TAG.

Nonhalogenated forms of mEndoG were expressed in BL21 Codon+ cells. Cells were grown at 37° C. in 1 liter of NZCYM media in the presence of the appropriate antibiotics (kanamycin and chloramphenicol) and 1% (w/v) dextrose. Protein expression was induced with 1 mM isopropyl β-D-1-thiogalactopyranoside (IPTG) when cells reached growth measured as 0.6 OD₆₀₀. The expression cultures continued to grow with shaking at 37° C. for 90 minutes or 48 hours at 16° C. Cells were centrifuged into pellets and stored at −20° C.

Constructs for mEndoG containing the unnatural ^{mX}Y amino acids were expressed in BL21 Codon+ cells by co-transforming the ^{mX}Y-mEndoG vector with the pdule-Mb-haloTyrRS-C6 plasmid, which encodes MbtRNACUA and 3-halo-L-Tyr amino acyl-tRNA synthetase, and that was altered to include ampicillin resistance (amp^{R}). Cells were grown at 37° C. in 1 liter of NZCYM media with kanamycin (52 mg), chloramphenicol (36 mg), and ampicillin (36 mg). Protein expression was induced with addition of 1 mM IPTG and 1 mM ^{mX}Y (^{mI}Y or ^{mCI}Y) at 0.6 OD₆₀₀. The culture solution then continued to shake for 4.5 hours at 25° C. before centrifugation and storing the pellet at −20° C.

Protein Purification. Cell pellets after protein expression were resuspended in ~25 mL of 50 mM Tris buffer at pH 8.0, 50 mM NaCl, 1 mM MgCl₂, 700 μL/L α-mercaptoethanol (BME) and 0.5% w/v glycerol, then incubated with 3 mL of 0.5 M EDTA and 10 μL of the detergent nonyl phenoxypolyethoxylethanol prior to sonication. The sonicated solution was then centrifuged at 15,000 rpm and the supernatant filtered through a 0.22 μm filter. The filtered solution was passed through an MBP-Trap column and eluted with 20 mM maltose. The eluate was collected and incubated with tobacco etch virus (TEV) protease for 16 hours at 4° C. to remove the MBP tag. The cleaved protein solution was passed through a Heparin column and the mEndoG construct eluted with 1 M NaCl. The eluate was concentrated to 1 mL then passed through a Sephracyl S-100 HR gel filtration column. The final sample was concentrated and stored at −80° C. until used.

DNA Substrate Preparation. Holliday junctions were assembled by annealing four complementary DNA oligonucleotides, one strand modified at the 5′-end with the Cy5 and a second with the fluorescein FITC fluorophores (sequences listed below). Oligonucleotides, purchased in HPLC purified form from Integrated DNA technologies (Coralville, IA), were dissolved in nano-pure water and junction buffer (2×TBE buffer at pH 7.8 plus 0.1M MgCl₂, 0.2M NaCl) to a concentration of 7 μM. The DNA heated to 90° C. for 20 minutes, followed by a 2-3 hour slow cooldown to room temperature. The annealed DNA was separated using a native polyacrylamide gel electrophoresis. Gel slices containing the junction DNAs were excised, crushed, and resuspended in 1X junction buffer. Duplex forms of the DNA result from mis-assembly or self-disassembly of the junction sequences.

The DNA sequences annealed as junctions are the following (^{5hm}C: 5-hydroxymethylcytosine):

(SEQ ID NO: 1)
CCGTACGGGG(5hmC)CAGGCCTCG
(exemplified sequence
includes a 5′ Cy5 tag);
(SEQ ID NO: 2)
GGGTTCCTGGCCCCGTACGG;
(SEQ ID NO: 3)
CGAGGCCTGGCCCCGGCTGC;
and
(SEQ ID NO: 4)
GCAGCCGGGGCCAGGAACCC
(exemplified sequence includes a 5′ FITC tag).

Nuclease Assays for Activity. DNA substrates for nucleases assays (prepared as described above) were diluted to 45-50 nM in reaction buffer (20 mM Tris buffer at pH 7.5, 0.4% glycerol, 10 mM BME, 0.1% Triton X-100) and incubated with 0.5 μM enzyme for varying time points (the buffer was altered for pH and EDTA content as necessary for the respective assays). Reactions were quenched with 1.5 μM proteinase K and 0.02% sodium dodecyl sulfate (SDS). The quenched reaction mediums were separated by native or denaturing polyacrylamide gel electrophoresis (PAGE) and imaged using a Typhoon FLA 9500 gel imager (GE Healthcare). The PAGE images were scanned and quantified using the NIH ImageJ program.

Kinetics of the enzyme catalyzed reactions were treated as pseudo-first order reactions and fitted as single-exponential functions. The resulting apparent rate constants (k_{APP}) were measured at least twice for each condition through a minimum of two, typically three independent replicate experiments.

Fluorescence Polarization Assays for Affinity. The affinity of various mEndoG constructs for the Cy5-labeled DNA junctions were assayed by determining their dissociation constants (K_{D}) from fluorescence polarization (FP) measurements using a PerkinElmer 1420 Multilabel counter plate reader and FP curves analyzed using the program KaleidaGraph. Assays were performed in solutions containing 20 nM FITC-labeled DNA plus enzyme concentrations ranging from 0.1 to 10 μM in 50 mM TRIS buffer at pH 8.0, plus 50 mM NaCl and 1 mM MgCl₂. Under these solution conditions, the enzymes do not show significant cleavage of the DNA (WT-mEndoG cleaves <2% of the DNA after 2 hours incubation).

Molecular Models of mEndoG Constructs. Initial models for the various halogenated and nonhalogenated constructs of the mEndoG enzyme were constructing starting with the crystal structure of the WT enzyme (PDB ID 6NJU) using the BUILD functions in the program PYMOL. The PYMOL SCULPTING function was used to remove steric clashes, but no additional higher-level modeling was performed on the structures in order to eliminate any bias in the modeling.

Models for Mg²⁺ coordination complex in WT-mEndoG. Models for the mEndoG metal site (Mg²⁺-N128-E136) were constructed by placing the atoms of acetamide and acetate around the Mg²⁺ cation at equivalent positions seen in the WT-mEndoG crystal structure. In addition, waters W1 to W4 in FIG. 1C were placed according to their positions in the crystal structure to construct the model with four solvent molecules (W5 was not included, since it would have been replaced by an oxygen from the DNA phosphoribose backbone). The model for the three-water complex included W1-3, while the two-water complex included only W1 and W2. Hydrogens were added to all molecules including the waters. The positions of the hydrogen atoms were geometry optimized using density functional theory (DFT) optimization, while holding the position of all heavy atoms fixed in their respective positions. ESP surface calculations were performed on these hydrogen optimized models.

Models for H-bonded network in ^{mX}Y-mEndoG: Ortho-halophenol molecules were used as minimal models for the aromatic side chains of the respective iodo- and chloro-meta-halotyrosine amino acids. As described above, hydrogens were added to models and their geometries optimized by DFT calculations, while holding all heavy atoms fixed. The hydrogens of the hydroxyl substituents in these models were oriented away from the halogens since it was determined that this OH is likely not the H-bond donor of the HBeXB. Calculations with the OH oriented towards the halogens increased the ESP values by +1 to +8 kcal/mol of each model but did not affect the overall trends observed from the calculations. The simplest models were those of the o-halophenol to mimic the halogenated tyrosyl side chain with no enhancement of its X-bonding potential from an H-bond donor.

To build the H-bonded HBeXB network, the o-halophenol and an acetamide molecule were used to mimic N103, with all atoms placed in positions as seen in the crystal structure of mEndoG, with the carbonyl oxygen is oriented towards the halogen. In this and all subsequent models, the atomic positions of the carbon equivalent to the Ca of the enzyme backbone were held fixed. The positions of the equivalent Cb atom of the acetamide was fixed in place, while the coordinates of the remainder of the molecule were geometry optimized by DFT calculations. The H-bonded o-halophenol was constructed by rotating the amide to point the NH₂ group towards the halogen and, again, the acetamide component geometry optimized with the equivalent Cb atom fixed.

Next, an imidazole molecule positioned to model the neutral H104 . . . N103 . . . ^{mX}Y136 network in the crystal structure was added, initially with the Nd equivalent nitrogen protonated and the Ne unprotonated. Again, the positions of atoms of the N103 and H104 mimics were geometry optimized by DFT while holding the Cb of the acetamide and the imidazole components fixed. The remainder of the atoms were allowed to rotate around the Ca-Cb bonds.

Quantum Mechanical (QM) Calculations of Electrostatic Potentials (ESPs). Minimum molecular models were constructed from small molecules that mimic the catalytic components of the WT-mEndoG and the ^{mX}Y-mEndoG constructs. Models did not include any atoms from the docked DNA substrate since these positions are uncertain. Electrostatic potentials for each model system were calculated at the Møller-Plesset second-order (MP2) level of theory using the program GAUSSIAN. Calculations on non-iodine models were performed with the 6-311+g (d) basis set, with an implicit solvent model having a dielectric constant D=2, shown to be appropriate for electrostatic type interactions in active sites of proteins, including those that bind nucleic acids. Calculations on the iodinated models applied an EMSL basis set exchange.

1. A method of modulating the catalytic activity of a modified endonuclease, the method comprising:
(a) contacting the modified endonuclease with a target nucleic acid in a reaction medium, wherein the modified endonuclease comprises a halogenated amino acid substituted at a position corresponding to a divalent metal ion-coordinating residue of a parent endonuclease and the halogenated amino acid forms a halogen bond with the target nucleic acid; and
(b) adjusting the reaction medium to a pH where the modified endonuclease cleaves the target nucleic acid.

2. The method of claim 1, wherein the halogenated amino acid is a halogenated tyrosine.

3. (canceled)

4. The method of claim 2, wherein the halogenated tyrosine is meta-iodotyrosine.

5. The method of claim 1, wherein the parent endonuclease is endonuclease G (EndoG) or a homolog or ortholog thereof.

6. The method of claim 1, wherein the pH where the modified endonuclease cleaves the target nucleic acid is below 7.0 or above 8.0.

7. The method of claim 1, wherein the method further comprises adjusting the pH of the reaction medium to a pH which reduces the catalytic activity of the modified endonuclease.

8. (canceled)

9. The method of claim 7, wherein the pH which reduces the catalytic activity of the modified endonuclease is 7.0 to 8.0.

10. (canceled)

11. The method of claim 1, wherein the pH of the reaction medium is below 7.0.

12. The method of claim 11, wherein the reaction medium is substantially free of divalent metal cofactor and/or the modified endonuclease retains catalytic activity in the presence of a metal chelator at a concentration sufficient to substantially inactivate the parent endonuclease.

13. (canceled)

14. The method of claim 1, wherein the pH of the reaction medium is above 8.0.

15. The method of claim 14, wherein the reaction medium comprises a divalent metal cofactor.

16. (canceled)

17. A method for cleaving a target nucleic acid, the method comprising:
(a) providing a modified endonuclease comprising a halogenated amino acid substituted at a position corresponding to a divalent metal ion-coordinating residue of a parent endonuclease, wherein the halogenated amino acid forms a halogen bond with a target nucleic acid; and
(b) contacting the target nucleic acid with the modified endonuclease at a select pH where the modified endonuclease cleaves the target nucleic acid.

18. The method of claim 17, wherein the select pH is below 7.0 or above 8.0 and wherein the modified endonuclease exhibits reduced catalytic activity at a pH of 7.0 to 8.0 relative to activity at a pH below 7.0 or above 8.0.

19. The method of claim 18, wherein the select pH is from 5.5 to 7.0 or from 8.0 to 10.0.

20. (canceled)

21. The method of claim 17, wherein the method comprises adjusting the pH one or more times between a pH wherein the modified endonuclease exhibits reduced catalytic activity and the select pH.

22. The method of claim 17, wherein the select pH is below 7.0 and method does not comprise contacting the modified endonuclease and/or target nucleic acid with a divalent metal cofactor.

23-24. (canceled)

25. The method of claim 17, wherein the select pH is above 8.0 and the method further comprises contacting the modified endonuclease and/or target nucleic acid with a divalent metal cofactor.

26-27. (canceled)

28. The method of claim 17, wherein the halogenated amino acid is a halogenated tyrosine.

29. (canceled)

30. The method of claim 28, wherein the halogenated tyrosine is meta-iodotyrosine.

31-32. (canceled)

33. The method of claim 17, wherein the parent endonuclease is endonuclease G (EndoG) or a homolog or ortholog thereof.


SEQUENCE LISTING (4 sequences)

SEQ ID NO: 1 - DNA, 20 bp, synthetic construct
  source 1..20: mol_type = other DNA
  modified_base 11: mod_base = OTHER; note = 5-hydroxymethylcytosine
ccgtacggggccaggcctcg

SEQ ID NO: 2 - DNA, 20 bp, synthetic construct
  source 1..20: mol_type = other DNA
gggttcctggccccgtacgg

SEQ ID NO: 3 - DNA, 20 bp, synthetic construct
  source 1..20: mol_type = other DNA
cgaggcctggccccggctgc

SEQ ID NO: 4 - DNA, 20 bp, synthetic construct
  source 1..20: mol_type = other DNA
gcagccggggccaggaaccc